(19) United States
(12) Patent Application Publication
(10) Pub. No.: US 2025/0270681 A1
(43) Pub. Date: Aug. 28, 2025

(54) ZR-CU-AL ALLOY METALLIC GLASSES

(71) Applicant: VULKAM, GIÈRES (FR)

(72) Inventors: Alexis LENAIN, LA TRONCHE (FR); Léna VERCHÈRE, LA TRONCHE (FR)

(73) Assignee: VULKAM, GIÈRES (FR)

(21) Appl. No.: 18/858,355

(22) PCT Filed: May 5, 2023
(86) PCT No.: PCT/EP2023/062055

§ 371 Date: Oct. 21, 2024

(30) Foreign Application Priority Data
May 5, 2022 (FR) FR2204260

(51) Int. Cl. C22C 45/10

(52) U.S. Cl. CPC C22C 45/10; C22C 2200/02

(57) ABSTRACT

A metallic glass formed from an alloy having the elements: Zr from 45 to 68 atomic percent, and Cu less than 25 atomic percent; and Al having between 9 and 12 atomic percent; and Ti from 0.5 to 10 atomic percent; and Nb from 0.1 to 6 atomic percent; and other elements not more than 0.1% by weight each and not more than 0.5% by weight in total; and the total sum of the preceding elements being equal to 100% by weight in total; and the sum Zr+Nb+Ti is between 64 and 69 atomic percent. The invention also relates to a part made of metallic glass and to its manufacturing method.

### TECHNICAL FIELD

The invention concerns new zirconium-copper-aluminium (Zr—Cu—Al) alloy metallic glasses, more particularly such Zr—Cu—Al alloy metallic glasses comprising niobium, Nb, and having improved properties, particularly suitable for medical applications.

### PRIOR ART

The “amorphous metallic alloys” (AMAs) or “metallic glasses” have exceptional mechanical properties compared to their traditional crystalline counterparts: high elastic limit and hardness, high elastic deformation capacity, high resistance to fatigue, corrosion and abrasion. Long limited by manufacturing methods inducing geometries not very inclined to industrialization, AMA parts may now be obtained industrially via in particular a method comprising two successive steps: (i) the melting of a set of pure metal ingots defining the composition of the final material, then (ii) the extremely rapid cooling of the liquid mixture resulting in a solidification without the formation of crystals or, at the very least, whose amorphous phase is predominant compared to the crystalline phase. This method may then make it possible to produce parts of centimeter size with sub-micrometric geometric details and very high form factors, sometimes even in the absence of subsequent machining operation.

The novel properties of AMAs have naturally aroused a great interest in the design of microparts subjected to high mechanical stresses. This interest is now growing due to the need to miniaturize the systems in many sectors, particularly those of the medical or dental fields.

The health field has its own particularities, in particular the limitation or the exclusion of alloy elements likely to have toxicity for humans, an excellent resistance to corrosion, a high compromise of mechanical properties so that the microparts are not only resistant but sufficiently deformable so as not to break brittle and leave alloy residues in the body. In addition to these specific properties, the alloy must also have an excellent shaping capacity, therefore, in the present case, an excellent moldability for the industrial manufacture of the microparts.

Although AMAs are likely to have exceptional properties, they are also complex to produce. Indeed, each element composing an AMA interacts with the others and this in a different way depending on the content of each of these so-called alloy elements.

The ZrCuAl amorphous alloys comprising or not containing Ti have at first sight potentially interesting properties for the medical applications. Indeed, zirconium and titanium are likely to give the alloys a good resistance to corrosion and are also biocompatible. In addition, it is known that amorphous alloys can be obtained in the Zr—Cu—Al, Zr—Ni—Al and Zr—Co—Al systems. The use of Co is however not suitable for medical applications due to its carcinogenic nature. Moreover, the allergenic nature of Ni and the high viscosity of the Zr—Ni—Al alloys mean that they are not a priori suitable for such applications either and it is also difficult to manufacture good quality microparts with such alloys.

For example, U.S. Pat. No. 9,724,450 B2 discloses (Zr, Ti)ₐ(Ni,Cu,Fe)_{b}(Be,Al, Si,B), and Zrₐ(Nb, Ti)_{b}Cu_{c}Al_{d} alloys to carry out medical implants. However, this patent only explicitly discloses the Zr₄₇Ti₈Ni₁₀Cu_{7.5}Be_{27.5} and Zr_{56.2}Ti_{13.8}Nb_{5.0}Cu_{6.9}Be_{12.5} alloys containing beryllium, an element classified as toxic if ingested, causing skin irritations and allergies, fatal if inhaled, carcinogenic and also known to cause serious damage to organs through prolonged or repeated exposure according to the CLP Regulation (EC No 1272/2008).

Application 1 US2013/0032252 A1 also cites zirconium-based amorphous alloys for the biomedical applications, in particular ZrNbTiCuAl alloys. However, none of the alloys cited are specifically characterized in this patent application and the present inventors have found that none of these alloys, taken together, have the properties necessary for real use in the medical field.

### Technical Problem

The known solutions do not allow to obtain a ZrCuAl-based metallic glass micropart having both a limitation or exclusion of alloy elements likely to have toxicity for humans, an excellent resistance to corrosion, a high compromise of mechanical properties so that the microparts are not only resistant but sufficiently deformable as well as an excellent shaping capacity.

Therefore, there is a need for a new ZrCuAl alloy system in order to solve the above disclosed problems.

### DISCLOSURE OF THE INVENTION

There is provided a metallic glass formed from an alloy comprising the elements:
Zr: proportion from 45 to 68 atomic percent, preferably from 48 to 65 atomic percent, and
Cu: proportion less than 25 atomic percent, preferably less than 24 atomic percent; and
Al: proportion comprised between 9 and 12 atomic percent, preferably from 9 to 11 atomic percent; and
Ti: proportion from 0.5 to 10 atomic percent, preferably from 2 to 8 atomic percent; and
Nb: proportion from 0.1 to 6 atomic percent, preferably from 0.5 to 4 atomic percent, more preferably from 1.5 to 3 atomic percent; and
other elements not more than 0.1% by weight each and not more than 0.5% by weight in total; and
the total sum of the proportions of said preceding elements being equal to 100% by weight in total; and the sum of the proportions of Zr+Nb+Ti is comprised between 64 and 69 atomic percent, preferably from 65 to 68 atomic percent.

According to another aspect, there is provided a part made of metallic glass as described above.

According to another aspect, there is also provided a method for manufacturing a part made of metallic glass as described above and comprising the following steps:
melting a mixture of metals to obtain an alloy,
molding the alloy obtained in a mold, optionally a mold comprising a sacrificial insert,
cooling the molded alloy with a cooling rate greater than the critical crystallization rate of the alloy, to obtain an amorphous alloy preform or a part made of amorphous alloy,
demolding the amorphous alloy preform or the amorphous alloy part, and, optionally, dissociating the sacrificial insert therefrom, preferably by chemical dissolution,
optionally, machining the amorphous alloy preform, preferably by laser machining, turning, bar turning and/or cylindrical grinding or centerless grinding, to obtain a part made of amorphous alloy according to a predetermined geometry,
optionally, carrying out at least one step of finishing the part made of amorphous alloy such as a surface texturing step, a chemical machining step and/or a chemical surface passivation treatment.

The characteristics disclosed in the following paragraphs may optionally be implemented. They may be implemented independently of each other or in combination with each other.

According to one arrangement, the metallic glass according to the invention comprises an amorphous phase fraction greater than 50%, preferably greater than 60%, more preferably still greater than 70% and even greater than 80%.

According to one possibility, the metallic glass according to the invention comprises a crystalline phase fraction strictly less than 50%, preferably strictly less than 40%, more preferably still strictly less than 30%, more preferably still strictly less than 20%.

The determination of the amorphous fraction of the metallic glass according to the invention will be explained in more detail below in the part of the description of the embodiments and illustrated in FIGS. 3 to 5.

According to an advantageous embodiment, the metallic glass is such that: Zr is comprised between 50 and 62 atomic percent, preferably from 55 to 60 atomic percent, more preferably from 58% to 60 atomic percent.

According to another embodiment, the metallic glass is such that the proportion of Cu is from 19 to 24 atomic percent, preferably from 20 to 24 atomic percent, more preferably from 21 to 24 atomic percent and; even more preferably from 22 to 24 atomic percent.

Advantageously, the metallic glass is such that the proportion of Ti is 3 to 8 atomic percent, preferably from 4 to 7 atomic percent and, more preferably still between 5 and 7 atomic percent.

Preferably, the metallic glass is selected from: Zr₅₉Cu₂₃Al₁₀Ti₆Nb₂, Zr₆₁Cu₂₃Al₁₀Ti₄Nb₂, Zr₆₁Ti₂Nb₄Cu₂₃Al₁₀, Zr₆₀Ti₄Nb₂Cu₂₄Al₁₀, Zr_{61.2}Ti_{4.9}Nb_{1.9}Cu_{22.9}Al_{9.1}, Zr₅₉Ti_{4.75}Nb₁Cu_{23.5}Al_{11.75}, Zr_{60.9}Ti₆Nb_{0.1}Cu₂₃Al₁₀, Zr₅₉Ti_{3.5}Nb_{1.75}Cu₂₄Al_{11.75}, Zr₅₈Ti₇Nb_{0.4}Cu_{24.5}Al_{10.1}, Zr₅₇Ti_{4.6}Nb_{2.5}Cu₂₄Al_{11.9}, Zr_{60.25}Ti_{4.5}Nb_{1.5}Cu₂₂Al_{11.75}, Zr₆₅Ti_{1.8}Nb_{0.8}Cu_{23.1}Al_{9.3}, and Zr_{61.95}Ti_{4.1}Nb_{2.7}Cu₂₂Al_{9.25}.

According to one embodiment, the part made of metallic glass has a critical thickness greater than or equal to 2 mm, preferably greater than or equal to 3 mm and, even more preferably greater than or equal to 5 mm.

The critical thickness is a characteristic well known to those skilled in the art, it is notably described in the document “Alliages métalliques amorphes; Yannick Champion; Techniques de l'ingénieur; Ref.: M4025 V1 and notably in chapter 2 which deals with the preparation of the amorphous metallic materials and notably the cooling rate and the amorphization capacity of these materials—point 2.1 (pages M 4 025-7 and following)”. This review was published in June 2011 then revalidated in October 2017 and perfectly reflects the general knowledge of those skilled in the art. As will be seen later, the measurement of the critical thickness performed within the scope of the invention uses the measurement method described in the article above.

According to one possibility, the critical thickness of the part made of metallic glass is determined by successive moldings of plates of the same surface area and of different thicknesses, molded from the liquid state under predefined conditions.

According to one arrangement, molding comprises melting the alloy, arranging the alloy in a mold followed by cooling the alloy.

According to other characteristics, the method for determining the critical thickness includes one or more of the following optional characteristics considered alone or in combination:
The surface area of the plates is approximately 2 cm².
The melting temperature of TI+150° C. with TI, the liquidus temperature of the alloy (in ° C.).
The mold is made of CuC1 type copper.
The maximum cooling temperature is approximately twenty degrees Celsius (20° C.).
The alloy is produced and molded under an inert, high-purity atmosphere (e.g. under argon grade 6.0) or under secondary vacuum (pressure <10-4 mbar).
The molding is carried out under a pressure of 20 MPa.
After molding, the plates are cut in order to obtain a slice, that is to say a longitudinal section of the plate, of different thicknesses.
The slices obtained are analyzed by X-ray diffraction to determine whether they have an amorphous or crystalline structure. The critical thickness is then determined as being the maximum thickness for which the structure is “totally amorphous” in the sense that the X-ray diffraction analysis of the alloy does not reveal a crystallinity peak.

According to one embodiment, the part made of metallic glass has a compromise of mechanical properties, evaluated according to a 3-point bending test, such that:
the elastic limit, σel, is greater than 1500 MPa, preferably greater than 1525 MPa, more preferably greater than 1550 Mpa; and
the plastic contribution to deflection, fp, is greater than 2 mm, preferably greater than or equal to 2.1 mm, more preferably greater than or equal to 2.2 mm; and/or
the percent of tests for which the deflection at break, fr, exceeds a value corresponding to twice the thickness of the specimen is greater than or equal to 80%, preferably greater than or equal to 90%, more preferably equal to 100%.

According to one possibility, the specimen is the part made of metallic glass according to the invention.

According to one arrangement, the 3-point bending test is carried out in the thickness direction of the part made of metallic glass of the invention.

According to one possibility, the elastic limit, σel, is calculated by applying the following formula 1:

$$σ el = \frac{3 \times Fe \times L}{2 \times b \times {h}^{2}}$$

with L being the length between the supports, for example L=10 mm, b being the width of the part, for example b=10 mm, h being the thickness of the part, for example h=1 mm, Fe being calculated according to the following formula 2:

$$Fe = 2 F max / 3$$

with Fmax: the maximum force value recorded at the force plateau of the part, and for example the crosshead speed v is 0.005 mm/s.

According to one arrangement, the plastic contribution to deflection, fp, of the part made of metallic glass is calculated according to the following formula 3:

$$fp = fr - fe$$

With fe: the deflection reached at a force level corresponding to Fe, i.e. force 2Fmax/3; and fr being the value of the deflection at break of the part.

According to another embodiment, the part made of metallic glass has a resistance to corrosion, evaluated according to the ISO 10271:2020 standard, such that the width of the passivation plateau ΔE is greater than 0.20 V/ECS, preferably greater than or equal to 0.30 V/ECS, more preferably greater than 0.45 V/ECS.

The resistance to corrosion of the samples is evaluated following the protocol defined in the ISO 10271:2020 standard for oral medicine.

According to one possibility, the resistance to corrosion of the metallic glass part samples is measured according to the steps of:
preparing the samples,
arranging the samples in a corrosive environment,
measuring the free potential E_{OCP} of the sample for a predetermined duration,
carrying out an intensity-potential curve at a given speed from a given potential until the current reaches a few dozen times the value of the pitting current,
detecting the pits, and
determining the corrosion potential Ecor.

According to one arrangement, the width of the passivation plateau ΔΕ is calculated as follows: ΔΕ=E_{piq}-E_{cor}; with E_{piq} the first pitting potential.

According to one possibility, the preparation of the samples comprises a step of polishing until to obtain a “mirror-polished” surface.

For example, the samples are polished using SiC paper and then a diamond suspension to a grain size of 1 μm, resulting in a “mirror polished” surface.

According to one possibility, the preparation of the samples also comprises after the polishing step a step of degreasing the samples, for example with acetone, then optionally a cleaning step, for example ultrasonically with ethanol, then with distilled water.

According to one arrangement, the corrosive environment in which the samples are arranged is obtained by preparing a 9 g/L NaCl solution at pH 7.4±0.1 buffered using a 4% NaOH solution and a 1% lactic acid solution.

According to one possibility, the corrosion test is carried out at a temperature of 37° C.±1° C.

According to one possibility, said solution is deaerated by bubbling argon for at least 30 minutes before arranging the samples.

According to one arrangement, a low bubbling is maintained during the test.

The measurement duration of the free potential E_{OCP} of the sample is approximately 2 hours.

The intensity-potential curve is carried out at a speed of +1 mV/s from E_{OCP}-150 mV until the current reaches 100 times the value of the pitting current.

According to one possibility, the samples are then rinsed, dried and viewed again under optical microscope to detect the pits.

According to one arrangement the corrosion potential E_{cor} is determined by the potential value for which the current is zero on the intensity-potential curve.

Advantageously, the part made of metallic glass is chosen from: all or part of a surgical or microsurgical instrument, all or part of a dental instrument, all or part of a suture device, all or part of an implant, in particular a dental, acoustic or orthopedic implant.

### BRIEF DESCRIPTION OF THE DRAWINGS

Other characteristics, details and advantages of the invention will appear on reading the detailed description below, and on analyzing the appended drawings, in which:

FIG. 1 represents a 3-point bending curve, obtained during mechanical tests, allowing to evaluate the elastic limit, σel, the deflection at break, fr, and the plastic contribution to deflection, fp, of the samples made of amorphous metallic alloy.

FIG. 2 represents a polarization curve obtained according to the corrosion test described in the ISO 10271:2020 standard and allowing in particular to evaluate the width of the passivation plateau ΔE of the samples made of amorphous metallic alloy.

FIG. 3 represents a XRD analysis of an amorphous metallic alloy.

FIG. 4 represents a XRD analysis of a partially amorphous metallic alloy.

FIG. 5 represents a XRD analysis of a crystalline metallic alloy.

### DESCRIPTION OF THE EMBODIMENTS

The drawings and the description below contain, for the most part, elements of a certain character. They may therefore not only serve to better understand the present invention but also contribute to its definition, if necessary. In the above, the following definitions should be clarified.

Here, the term “metallic glass” or “amorphous metallic alloy” or “AMA” means metals or metallic alloys that are not crystalline, that is to say whose atomic distribution is mainly random. Nevertheless, it is difficult to obtain a one hundred percent amorphous metallic alloy because there most often remains a fraction of the material that is crystalline in nature. This definition can therefore be generalized to metals or metallic alloys that are partially crystalline and which, therefore, contain a fraction of crystals, as long as the amorphous fraction is predominant compared to the crystalline fraction. The metallic glasses according to the present invention have an amorphous phase fraction greater than 50%, preferably greater than 60%, more preferably still greater than 70% and even greater than 80%.

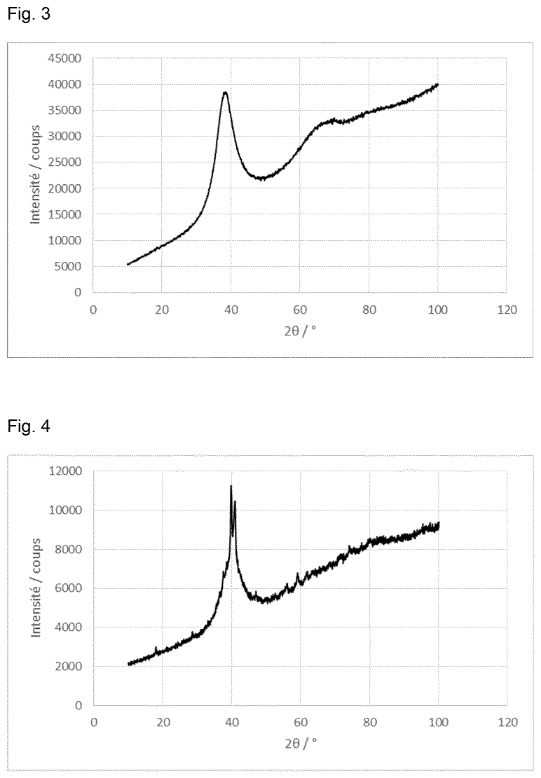

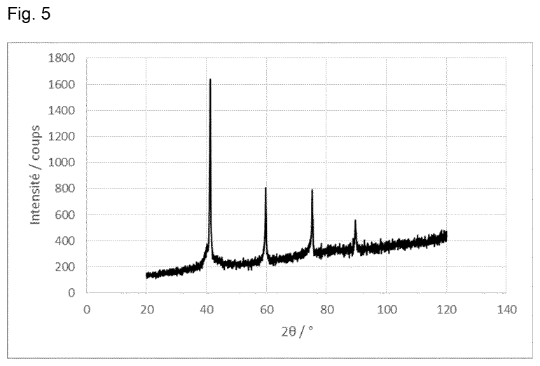

It is specified here that a metallurgical structure is said to be “totally amorphous” within the meaning of the present invention when an X-ray diffraction analysis as described below does not reveal any crystallization peak. A metallurgical structure is said to be “partially amorphous” within the meaning of the present invention when an X-ray diffraction analysis as described below reveals a few crystallization peaks. Unless otherwise specified, the term “amorphous” is used both for alloys said to be “totally amorphous” and for alloys said to be “partially amorphous” within the meaning of the invention. Such an evaluation of the amorphous nature of a metallic alloy is detailed in the article Cheung et al., 2007 (Cheung et al. (2007) “Thermal and mechanical properties of Cu—Zr—Al bulk metallic glasses)” doi: 10.1016/j.jallcom.2006.08.109). It allows an average analysis to be made on a surface and to overcome the few inevitable metallurgical defects, while analyzing only the crystals of significant size, that is to say greater than a few nanometers and/or in significant quantity. FIGS. 3, 4 and 5 represent a XRD analysis as described previously. These figures show the intensity of the diffracted beam as a function of the angle between the incident beam and the diffracted beam. FIG. 3 is a XRD analysis of a metallic alloy in the “totally amorphous” state, the amorphous fraction being very much predominant compared to the crystalline fraction. FIG. 4 is a similar analysis carried out on an alloy in the “partially amorphous” state, the amorphous fraction being predominant compared to the crystalline fraction. In this figure, we find the characteristic bump of the amorphous structures, but with the presence of peaks as well. FIG. 5 is a similar analysis carried out on a crystalline alloy, the crystalline fraction being predominant compared to the amorphous fraction. In this FIG. 5, the characteristic bump of AMAs is not present and the crystallinity peaks are clearly visible.

The terms “critical thickness” (denoted ec) of a specific amorphous metallic alloy mean the maximum limiting thickness below which the metallic alloy has a “totally amorphous” metallurgical structure or beyond which it is no longer possible to obtain a “totally amorphous” metallurgical structure, when the metallic alloy is molded from a liquid state and is subjected to a rapid cooling such that the transfer of the heat inside the metallic alloy is optimal. More specifically, the critical thickness is determined by successive moldings of plates of approximately 2 cm² and of different thicknesses, molded from the liquid state under the following conditions:
The alloy is melted at a temperature of TI+150° C. with TI, the liquidus temperature of the alloy (in ° C.);
The alloy is molded in a mold made of CuC1 type copper and cooled to a maximum temperature of approximately twenty degrees Celsius (20° C.).
The alloy is produced and molded under an inert and high-purity atmosphere (e.g. under argon grade 6.0) or under secondary vacuum (pressure <10-4 mbar). The alloy is molded with a system allowing the application of a pressure differential to facilitate the molding of the alloy and to ensure an intimate contact between the alloy and the walls of the mold in order to ensure a rapid cooling of the alloy. The molding step may be carried out under a pressure of 20 MPa. This overpressure application system may be mechanical (piston) or gaseous.
After molding, the plates are cut in order to obtain a slice, that is to say a longitudinal section of the plate, of different thicknesses.
The obtained slices are analyzed by X-ray diffraction to determine whether they have an amorphous or crystalline structure. The critical thickness is then determined as being the maximum thickness for which the structure is “totally amorphous” in the sense that the X-ray diffraction analysis of the alloy does not reveal a crystallinity peak.

According to the present description, the elastic limit, σel, and the plastic contribution to deflection, fp, are evaluated as follows.

The mechanical tests are carried out on a mechanical testing machine DY34 (Adamel Lhomargy). These are 3-point bending tests in the thickness direction of the sample.

The parameters of the test are as follows:
Length between supports L=10 mm
Sample width b=10 mm
Sample thickness h=1 mm
Sample length I=15 mm
Crosshead speed v=0.005 mm/s

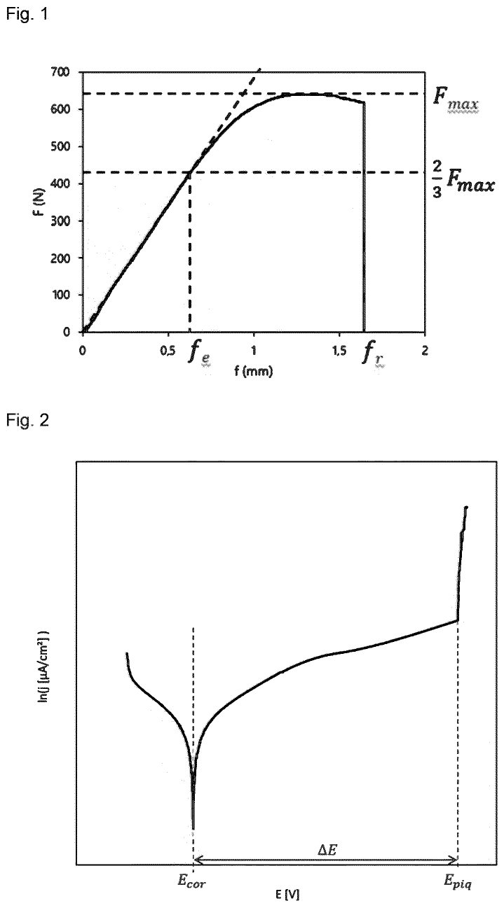

The 3-point bending curve has a first linear elastic part, during which the sample deforms elastically, then a plastic plateau, during which the deformation is plastic (see FIG. 1).

The elastic limit, σel, is calculated according to the following formula 1:

$$σ el = \frac{3 \times Fe \times L}{2 \times b \times {h}^{2}}$$

where Fe is calculated according to the following formula 2:

$$Fe = 2 F max / 3$$

with Fmax: the maximum force value recorded at the force plateau.

The plastic contribution to deflection, fp, is calculated according to the following formula 3:

$$fp = fr - fe$$

with: -fe is the deflection reached at a force level corresponding to Fe, i.e. force 2Fmax/3; and
fr is the deflection at break.

The test is stopped when the specimen breaks or the deflection reaches a value of 2.5 mm.

The number of tests for which the deflection at break, fr, exceeds a value corresponding to twice the thickness of the specimen (fr>2*h) is counted.

The alloys for which the percent of tests where the deflection at break, fr, exceeds a value corresponding to twice the thickness of the specimen (fr>2*h) is high and, more particularly, equal to 100%, have a remarkable and reproducible plasticity, which is essential for the intended applications.

FIG. 1 illustrates a 3-point bending curve obtained according to the test described above.

Each alloy has its own crystallization temperature Tx and glass transition temperature Tg. These temperatures are measured using a scanning calorimeter (DSC) at a rise rate of 20° C./min. The temperatures Tg and Tx are then extracted from the DSC curves.

For each alloy, it is thus possible to determine the difference ΔTx between the crystallization temperature Tx and the glass transition temperature Tg, i.e. ΔTx=Tx−Tg.

According to this description, the resistance to corrosion is evaluated as follows: the samples are polished using SiC paper and then a diamond suspension to a particle size of 1 μm, to obtain a “mirror polished” surface. They are then observed under an optical microscope. Just before the corrosion test, the samples are degreased with acetone and then cleaned ultrasonically with ethanol, then with distilled water. In accordance with the ISO 10271:2020 standard, the corrosion test is carried out in a 9 g/L NaCl solution at pH 7.4±0.1 buffered with a 4% NaOH solution and a 1% lactic acid solution, at a temperature of 37° C.±1° C. The solution is deaerated by bubbling argon for at least 30 min. A low bubbling is maintained during the test. The corrosion test consists of measuring the free potential E_{OCP} of the sample for 2 hours, then carrying out an intensity-potential curve at a speed of +1 mV/s from E_{OCP}-150 mV until the current reaches 100 times the value of the pitting current. The samples are then rinsed, dried and observed again under optical microscope to detect the pits. The corrosion potential E_{cor} is the potential value for which the current is zero on the intensity-potential curve. The width of the passivation plateau ΔE is calculated as follows: ΔE=E_{piq}-E_{cor}; with E_{piq} the first pitting potential.

FIG. 2 illustrates a polarization curve obtained according to the corrosion test previously described.

As previously indicated, the AMAs known until now, in particular those whose major elements are zirconium, copper and aluminium, have a low resistance to corrosion and/or a compromise of mechanical properties, in particular for properties such as their elastic limit and their plastic contribution to deflection, not optimized and/or a lower processability making their industrialization complex.

Against all expectations, the present inventors were able to overcome these problems and reference is now made to the amorphous metallic alloy, also referred to as “metallic glass”, which is the subject of the present invention.

The present metallic glass is thus formed from an alloy comprising:
Zr: from 45 to 68 atomic percent, preferably from 48 to 65 atomic percent; and
Cu: less than 25 atomic percent, preferably less than 24 atomic percent; and
Al: comprised between 9 and 12 atomic percent, preferably 9 to 11 atomic percent; and
Ti: from 0.5 to 10 atomic percent, preferably from 2 to 8 atomic percent; and
Nb: from 0.1 to 6 atomic percent, preferably from 0.5 to 4 atomic percent, more preferably from 1.5 to 3 atomic percent; and
other elements not more than 0.1% by weight each and not more than 0.5% by weight in total; and
the total sum of said preceding elements being equal to 100% by weight in total; and the sum Zr+Nb+Ti is comprised between 64 and 69 atomic percent, preferably from 65 to 68 atomic percent.

The metallic glass is formed from an alloy comprising zirconium, Zr. More particularly, it comprises from 45 to 68 atomic percent of Zr, preferably from 48 to 65 atomic percent, more preferably Zr is comprised between 50 and 62 atomic percent, more preferably Zr is from 55 to 60 atomic percent, or even from 58% to 60 atomic percent. The Zr content of the alloy influences in particular the critical thickness, ec, of the alloy. More generally, the content of each alloy element must be specifically selected to obtain a metallic glass having a good vitrification capacity. It is in fact the overall formulation of the alloy which determines its critical thickness.

The metallic glass is formed from an alloy also comprising copper, Cu. More particularly, it comprises less than 25 atomic percent, preferably less than 24 atomic percent of Cu. Advantageously, the Cu content is such that Cu from 19 to 24 atomic percent, preferably from 20 to 24 atomic percent, more preferably from 21 to 24 atomic percent and; more preferably still from 22 to 24 atomic percent. The Cu content of the alloy influences in particular the critical thickness, ec, and the resistance to corrosion of the alloy. In addition, Cu is likely to be cytotoxic when it is present in large quantities. It is therefore essential for the intended applications to limit its content in the alloy.

The metallic glass is formed from an alloy also comprising titanium, Ti. The Ti content is such that: Ti from 0.5 to 10 atomic percent, preferably from 2 to 8 atomic percent, more preferably from 3 to 8 atomic percent, more preferably still from 4 to 7 atomic percent or even between 5 and 7 atomic percent. The Ti content of the alloy influences in particular the critical thickness, ec, of the alloy.

The metallic glass is formed from an alloy also comprising aluminium, Al. The Al content is such that: Al is comprised between 9 and 12 atomic percent, preferably from 9 to 11 atomic percent. Against all expectations, it has been shown that such an Al content makes it possible in particular to obtain a metallic glass having an excellent compromise of mechanical properties, the elastic limit, σel, and the plastic contribution to deflection, fp, being however properties known to be antinomic. Furthermore, such a selected Al content makes it possible to obtain a metallic glass having an excellent plasticity which is entirely reproducible; which results in particular in specimens which resist a deflection greater than twice the thickness of the specimen (fr>2*h).

The metallic glass is formed from an alloy also comprising niobium, Nb. The Nb content is such that: Nb from 0.1 to 6 atomic percent, preferably from 0.5 to 4 atomic percent, more preferably from 1.5 to 3 atomic percent. Against all expectations in this Zr—Cu—Al alloy system comprising Ti and Nb, it has been demonstrated that such a selected Nb content makes it possible in particular to improve the resistance to corrosion of the metallic glass, in particular its passivity, ΔE.

The metallic glass is formed from an alloy that may also comprise other elements, also called “residual impurities”, such as in particular oxygen, carbon, phosphorus and/or other metallic elements than those mentioned above. These residual impurities may also be any other element(s) not added voluntarily during the mixing of metals to obtain the alloy billet. The impurity content of the alloy, as a percent by weight, is not more than 0.1 each and not more than 0.5 in total. More preferably, this content is, as a percent by weight, not more than 0.05 each and not more than 0.2 in total. Preferably, the alloy comprises less than 250 ppm (parts per million) by weight, more preferably less than 200 ppm by weight and even more preferably less than 150 ppm by weight of each of these impurities.

The alloy of the metallic glass also comprises a selected content of Zr, Nb and Ti such that the sum Zr+Nb+Ti is comprised between 64 and 69 atomic percent, preferably from 65 to 68 atomic percent. Such a selection of the contents of Zr, Nb and Ti makes it possible in particular to obtain a metallic glass having an excellent resistance to corrosion and a very good vitrification capacity demonstrated in particular by a high critical thickness, ec.

According to a preferred embodiment, the amorphous metallic alloy is selected from: Zr₅₉Cu₂₃Al₁₀Ti₆Nb₂, Zr₆₁Cu₂₃Al₁₀Ti₄Nb₂, Zr₆₁Ti₂Nb₄Cu₂₃Al₁₀, Zr₆₀Ti₄Nb₂Cu₂₄Al₁₀, Zr_{61.2}Ti_{4.9}Nb_{1.9}Cu_{22.9}Al_{9.1}, Zr₅₉Ti_{4.75}Nb₁Cu_{23.5}Al_{11.75}, Zr_{60.9}Ti₆Nb_{0.1}Cu₂₃Al₁₀, Zr₅₉Ti_{3.5}Nb_{1.75}Cu₂₄Al_{11.75}, Zr₅₈Ti₂Nb_{0.4}Cu_{24.5}Al_{10.1}, Zr₅₇Ti_{4.6}Nb_{2.5}Cu₂₄Al_{11.9}, Zr_{60.25}Ti_{4.5}Nb_{1.5}Cu₂₂Al_{11.75}, Zr₆₅Ti_{1.8}Nb_{0.8}Cu_{23.1}Al_{9.3}, and Zr_{61.95}Ti_{4.1}Nb_{2.7}Cu₂₂Al_{9.25}.

The alloy as described above makes it possible, against all expectations, to obtain parts made of metallic glass having a completely exceptional compromise of properties as indicated above.

According to a preferred embodiment, the part made of metallic glass has a critical thickness greater than or equal to 2 mm, preferably greater than or equal to 3 mm and even more preferably greater than or equal to 5 mm.

The part made of metallic glass may also have a compromise of mechanical properties, evaluated according to a 3-point bending test, such that:
the elastic limit, σel, is greater than 1500 MPa, preferably greater than 1525 MPa, more preferably greater than 1550 MPa and more preferably still greater than 1565 MPa; and
the plastic contribution to deflection, fp, is greater than 2 mm, preferably greater than or equal to 2.1 mm, more preferably greater than or equal to 2.2 mm; and/or
the percent of tests for which the deflection at break, fr, exceeds a value corresponding to twice the thickness of the specimen is greater than or equal to 80%, preferably greater than or equal to 90%, more preferably equal to 100%.

The part made of metallic glass is also likely to have a resistance to corrosion, evaluated according to the ISO 10271:2020 standard, such that the width of the passivation plateau ΔE is greater than 0.20 V/ECS, preferably greater than or equal to 0.30 V/ECS, more preferably greater than 0.45 V/ECS, and more preferably still greater than or equal to 0.50 V/ECS.

Such a part made of metallic glass may in particular be obtained according to the manufacturing method comprising the following steps:
melting a mixture of metals to obtain an alloy,
molding the obtained alloy in a mold, optionally a mold comprising a sacrificial insert,
cooling the molded alloy with a cooling rate greater than the critical crystallization rate of the alloy, to obtain an amorphous alloy preform or a part made of amorphous alloy,
demolding the amorphous alloy preform or the amorphous alloy part, and, optionally, dissociating the sacrificial insert from the latter, preferably by chemical dissolution,
optionally, machining the amorphous alloy preform, preferably by laser machining, turning, bar turning and/or cylindrical grinding or centerless grinding, to obtain a part made of amorphous alloy according to a predetermined geometry,
optionally, carrying out at least one step of finishing the part made of amorphous alloy such as a surface texturing step, a chemical machining step and/or a chemical surface passivation treatment.

Preferably, the steps described above are carried out successively in the described order.

Advantageously, the molten metallic alloy may be shaped to obtain a billet. The billet is then melted, molded and cooled to obtain an amorphous alloy preform or a part made of amorphous alloy.

Advantageously, the alloy is molded in a mold comprising a sacrificial insert. Such a mold is described in particular in application WO 2020/128170 A1. It may in particular be a sacrificial insert made of silicon which will then be dissolved by selective chemical dissolution. Optionally, it may be necessary to carry out a step of removing excess material from the amorphous alloy preform or the amorphous alloy part, for example by machining.

Optionally, the amorphous alloy preform or the amorphous alloy part is machined, preferably by laser machining, turning, bar turning and/or cylindrical grinding or centerless grinding, to obtain a part made of amorphous alloy according to a predetermined geometry.

Optionally, it can be carried out at least one step of finishing the part made of amorphous alloy, such as a surface texturing step, a chemical machining step and/or a chemical surface passivation treatment. The surface texturing step is preferably carried out using a laser. Advantageously, the chemical machining step is carried out by electropolishing. According to a preferred embodiment, the chemical surface passivation treatment is carried out by chemical attack with HNO₃ in order to further increase the resistance to corrosion of the finished part.

### INDUSTRIAL APPLICATION

The invention may find application in particular in the medical or dental fields.

The parts made of metallic glass according to the invention are in particular suitable for the manufacture of all or part of a surgical or microsurgical instrument, all or part of a dental instrument, all or part of a suture device, all or part of an implant, in particular a dental, acoustic or orthopedic implant.

The invention is not limited to the above description alone and/or to examples 1 and 2 described below, but it encompasses all the variants that a person skilled in the art may envisage within the scope of the protection sought.

### EXAMPLES

### Example 1

Seven different compositions of metallic glass alloys, detailed in Table 1, were studied.

The primary alloys were produced by arc melting (T>2500° C.) of bulk fragments of high purity (>99.9%) basic elements under argon atmosphere using a Ti getter for the detection of any trace of harmful contamination. Each primary alloy was melted at least five times to ensure a high quality of chemical homogeneity. The alloy was injected into a mold to obtain a sample in the form of a 1 mm thick plate. This thickness, lower than the critical thickness, ensures that the structure obtained is amorphous. For all samples, the amorphous fraction is predominant compared to the crystalline fraction.

The resistance to corrosion was evaluated according to the test described above in the present description. The passivity (AE) of each alloy is reported in Table 1.

TABLE 1

| Entry | Composition | |
|---|---|---|
| No.: | (atomic percent)) | ΔE (V) |
| 1 | Zr₆₁Cu₂₅Al₁₂Ti₂ | 0.20 |
| 2 | Zr₆₁Cu₂₀Al₁₃Ti₄Nb₂ | 0.50 |
| 3 | Zr_{57.4}Cu₂₃Al₁₄Ti_{3.8}Nb_{1.9} | 0.23 |
| 4 | Zr_{62.2}Cu_{23.5}Al_{7.5}Ti_{5.4}Nb_{1.4} | 0.29 |
| 5 | Zr_{59.7}Cu₂₅Al₁₂Ti₂Nb_{1.3} | 0.44 |
| 6 | Zr₆₁Cu₂₃Al₁₀Ti₄Nb₂ | 0.30 |
| 7 | Zr₅₉Cu₂₃Al₁₀Ti₆Nb₂ | 0.50 |

The Zr₆₁Cu₂₅Al₁₂Ti₂ alloy, (entry 1) free of Nb, has a poor resistance to corrosion (Zr₆₁Cu₂₅Al₁₂Ti₂: ΔE<<0.30 V).

Adding Nb in the ZrCuAlTi alloy system (entries 2 to 7) can improve the corrosion of AMA in a corrosive environment.

### Example 2

Six different compositions of metallic glass alloys, detailed in Table 2, were studied.

The alloys were obtained according to the protocol described in example 1. For all samples, the amorphous fraction is predominant compared to the crystalline fraction.

The elastic limit σel, the plastic contribution to deflection, fp, and the percent of tests for which the deflection at break, fr, exceeds a value corresponding to twice the thickness of the specimen were evaluated using mechanical tests carried out in 3-point bending and described above in this description. At least 3 tests were carried out for each composition in order to ensure a good reproducibility of the results. The results are presented in Table 2.

TABLE 2

| | | | | Tests for |
|---|---|---|---|---|
| Entry | Composition | | | which |
| No.: | (atomic percent) | σel (MPa) | fp (mm) | fr >2*h |
| 1 | Zr_{62.2}Cu_{23.5}Al_{7.5}Ti_{5.4}Nb_{1.4} | 1491 ± 12 | 2.2 ± 0.0 | 100% |
| 2 | Zr_{59.7}Cu₂₅Al₁₂Ti₂Nb_{1.3} | 1610 ± 23 | 1.7 ± 0.6 | 67% |
| 3 | Zr_{57.4}Cu₂₃Al₁₄Ti_{3.8}Nb_{1.9} | 1687 ± 11 | 0.8 ± 0.5 | 0% |
| 4 | Zr₆₁Cu₂₀Al₁₃Ti₄Nb₂ | 1622 ± 18 | 1.7 ± 0.4 | 50% |
| 5 | Zr₆₁Cu₂₃Al₁₀Ti₄Nb₂ | 1555 ± 16 | 2.1 ± 0.2 | 100% |
| 6 | Zr₅₉Cu₂₃Al₁₀Ti₆Nb₂ | 1569 ± 13 | 2.2 ± 0.0 | 100% |

The Zr_{62.2}Cu_{23.5}Al_{7.5}Ti_{5.4}Nb_{1.4} alloy (entry 1) has an elastic limit, σel, too low for the intended application (σel<1500 MPa). The Zr_{57.4}Cu₂₃Al₁₄Ti_{3.8}Nb_{1.9}, Zr_{59.7}Cu₂₅Al₁₂Ti₂Nb_{1.3} and Zr₆₁Cu₂₀Al₁₃Ti₄Nb₂ alloys (entries 2 to 4) have a plastic contribution to deflection, fp, too low for the intended applications (fp<<2.00 mm) and the number of specimens that resist a deflection greater than twice the thickness of the specimen (fr>2*h) is less than or equal to 67% for these AMAs, which reflects both insufficient plasticity and reproducibility for the intended application.

The Zr₆₁Cu₂₃Al₁₀Ti₄Nb₂ and Zr₅₉Cu₂₃Al₁₀Ti₆Nb₂ alloys (entries 5 and 6) are the only ones to have an excellent compromise of mechanical properties; the elastic limit, σel, and the plastic contribution to deflection, fp, being however properties known to be antinomic.

### Example 3

Eleven different compositions of metallic glass alloys, detailed in Table 3, were studied.

The alloys were obtained according to the protocol described in example 1. For all samples, the amorphous fraction is predominant compared to the crystalline fraction.

The elastic limit gel, the plastic contribution to deflection, fp, and the percent of tests for which the deflection at break, fr, exceeds a value corresponding to twice the thickness of the specimen were evaluated using mechanical tests carried out in 3-point bending and described above in the present description. At least 3 tests were carried out for each composition in order to ensure a good reproducibility of the results. The results are presented in Table 3.

TABLE 3

| | | | | Tests for |
|---|---|---|---|---|
| Entry | Composition | | | which |
| No. | (atomic percent) | σel (MPa) | fp (mm) | fr >2*h |
| 1 | Zr₆₁Ti₂Nb₄Cu₂₃Al₁₀ | 1619 ± 12 | 2.1 ± 0.0 | 100% |
| 2 | Zr₆₀Ti₄Nb₂Cu₂₄Al₁₀ | 1614 ± 8 | 2.1 ± 0.0 | 100% |
| 3 | Zr_{61.2}Ti_{4.9}Nb_{1.9}Cu_{22.9}Al_{9.1} | 1578 ± 8 | 2.1 ± 0.0 | 100% |
| 4 | Zr₅₉Ti_{4.75}Nb₁Cu_{23.5}Al_{11.75} | 1648 ± 5 | 2.2 ± 0.0 | 100% |
| 5 | Zr_{60.9}Ti₆Nb_{0.1}Cu₂₃Al₁₀ | 1560 ± 11 | 2.2 ± 0.0 | 100% |
| 6 | Zr₅₉Ti_{3.5}Nb_{1.75}Cu₂₄Al_{11.75} | 1645 ± 21 | 2.1 ± 0.1 | 100% |
| 7 | Zr₅₈Ti₇Nb_{0.4}Cu_{24.5}Al_{10.1} | 1612 ± 3 | 2.2 ± 0.0 | 100% |
| 8 | Zr₅₇Ti_{4.6}Nb_{2.5}Cu₂₄Al_{11.9} | 1672 ± 32 | 2.2 ± 0.0 | 100% |
| 9 | Zr_{60.25}Ti_{4.5}Nb_{1.5}Cu₂₂Al_{11.75} | 1627 ± 5 | 2.2 ± 0.0 | 100% |
| 10 | Zr₆₅Ti_{1.8}Nb_{0.8}Cu_{23.1}Al_{9.3} | 1539 ± 1 | 2.1 ± 0.0 | 100% |
| 11 | Zr_{61.95}Ti_{4.1}Nb_{2.7}Cu₂₂Al_{9.25} | 1566 ± 11 | 2.1 ± 0.0 | 100% |

These eleven alloys all present an excellent compromise of mechanical properties: a high elastic limit, a high plasticity as well as a good reproducibility.

1. A metallic glass formed from an alloy comprising the elements:
Zr: from 45 to 68 atomic percent, and
Cu: less than 25 atomic percent; and
Al: comprised between 9 and 12 atomic percent; and
Ti: from 0.5 to 10 atomic percent; and
Nb: from 0.1 to 6 atomic percent; and
other elements not more than 0.1% by weight each and not more than 0.5% by weight in total; and
the total sum of the preceding elements being equal to 100% by weight in total; and
the sum Zr+Nb+Ti is comprised between 64 and 69 atomic percent.

2. The metallic glass according to claim 1, wherein Zr is comprised between 50 and 62 atomic percent.

3. The metallic glass according to claim 1, wherein Cu from 19 to 24 atomic percent.

4. The metallic glass according to claim 1, wherein Ti from 3 to 8 atomic percent.

5. The metallic glass according to claim 1 selected from: Zr₅₉Cu₂₃Al₁₀Ti₆Nb₂, Zr₆₁Cu₂₃Al₁₀Ti₄Nb₂, Zr₆₁Ti₂Nb₄Cu₂₃Al₁₀, Zr₆₀Ti₄Nb₂Cu₂₄Al₁₀, Zr_{61.2}Ti_{4.9}Nb_{1.9}Cu_{22.9}Al_{9.1}, Zr₅₉Ti_{4.75}Nb₁Cu_{23.5}Al_{11.75}, Zr_{60.9}Ti₆Nb_{0.1}Cu₂₃Al₁₀, Zr₅₉Ti_{3.5}Nb_{1.75}Cu₂₄Al_{11.75}, Zr₅₈Ti₇Nb_{0.4}Cu_{24.5}Al_{10.1}, Zr₅₇Ti_{4.6}Nb_{2.5}Cu₂₄Al_{11.9}, Zr_{60.25}Ti_{4.5}Nb_{1.5}Cu₂₂Al_{11.75}, Zr₆₅Ti_{1.8}Nb_{0.8}Cu_{23.1}Al_{9.3}, and Zr_{61.95}Ti_{4.1}Nb_{2.7}Cu₂₂Al_{9.25}.

6. The metallic glass according to claim 1, which comprises an amorphous phase fraction greater than or equal to 50%.

7. A part made of metallic glass wherein the metallic glass is according to claim 1, the part made of metallic glass having a critical thickness greater than or equal to 2 mm.

8. The part made of metallic glass according to claim 7, for which the critical thickness of the part made of metallic glass is determined by successive moldings of plates of the same surface area and of different thicknesses, molded from the liquid state under predefined conditions.

9. The part made of metallic glass according to claim 7, having a compromise of mechanical properties, evaluated according to a 3-point bending test, such that:
the elastic limit, σel, is greater than 1500 MPa; and
the plastic contribution to deflection, fp, is greater than 2 mm; and/or
the percent of tests for which the deflection at break, fr, exceeds a value corresponding to twice the thickness of the specimen is greater than or equal to 80%.

10. The part made of metallic glass according to claim 9 wherein the elastic limit, σel, is calculated by applying the following formula 1:
$$σ el = \frac{3 \times Fe \times L}{2 \times b \times {h}^{2}}$$
with L being the length between the supports, for example L=10 mm, b being the width of the part, for example b=10 mm, h being the thickness of the part, for example h =1 mm, Fe being calculated according to the following formula 2:
$$Fe = 2 F max / 3$$
with Fmax: the maximum force value recorded at the force plateau of the part, and for example the crosshead speed v is 0.005 mm/s.

11. The part made of metallic glass according to claim 9, wherein the plastic contribution to deflection, fp, of the part made of metallic glass is calculated according to the following formula 3:
$$fp = fr - fe$$
with fe: the deflection reached at a force level corresponding to Fe, i.e. force 2Fmax/3; and fr being the value of the deflection at break of the part.

12. The part made of metallic glass according to claim 7, having a resistance to corrosion evaluated according to the ISO 10271:2020 standard, such that the width of the passivation plateau ΔE is greater than 0.20 V/ECS, preferably greater than or equal to 0.30 V/ECS, more preferably greater than 0.45 V/ECS, the width of the passivation plateau ΔE being calculated as follows: ΔE=E_{piq}-E_{cor}; with E_{piq} the first pitting potential and E_{cor} the corrosion potential.

13. The part made of metallic glass according to claim 12, wherein the resistance to corrosion of the part made of metallic glass samples is evaluated according to the steps of:
preparing the samples,
arranging the samples in a corrosive environment,
measuring the free potential E_{OCP} of the sample for a predetermined duration,
carrying out an intensity-potential curve at a given speed from a given potential until the current reaches a few dozen times the value of the pitting current,
detecting the pits, and
determining the corrosion potential Ecor.

14. The part made of metallic glass according to claim 7 chosen from: all or part of a surgical or microsurgical instrument, all or part of a dental instrument, all or part of a suture device, all or part of an implant.

15. A method for manufacturing a part made of metallic glass according to claim 7 comprising the following steps:
melting a mixture of metals to obtain an alloy,
molding the obtained alloy in a mold, optionally a mold comprising a sacrificial insert,
cooling the molded alloy with a cooling rate greater than the critical crystallization rate of the alloy, to obtain an amorphous alloy preform or a part made of amorphous alloy,
demolding the amorphous alloy preform or the amorphous alloy part, and, optionally, dissociating the sacrificial insert from the latter, preferably by chemical dissolution,
optionally, machining the amorphous alloy preform to obtain a part made of amorphous alloy according to a predetermined geometry,
optionally, carrying out at least one step of finishing the part made of amorphous alloy such as a surface texturing step, a chemical machining step and/or a chemical surface passivation treatment.
